(19) United States
(12) Patent Application Publication
(10) Pub. No.: US 2015/0112210 A1
(43) Pub. Date: Apr. 23, 2015

(54) LUMEN BASED PRESSURE MEASUREMENT GUIDE WIRE SYSTEM FOR MEASURING PRESSURE IN A BODY LUMEN

(71) Applicant: ABBOTT CARDIOVASCULAR SYSTEMS INC., Santa Clara, CA (US)

(72) Inventor: William E. Webler, San Jose, CA (US)

(73) Assignee: ABBOTT CARDIOVASCULAR SYSTEMS INC., Santa Clara, CA (US)

(21) Appl. No.: 14/056,753

(22) Filed: Oct. 17, 2013

(51) Int. Cl. A61B 5/0215; A61B 5/00

(52) U.S. Cl. CPC A61B 5/0215; A61B 5/6851
USPC 600/486

(57) ABSTRACT

A medical system utilizes a guide wire with a fluid filled internal lumen and a pressure measurement system to measure blood pressure in a body vessel. The guide wire includes an internal lumen with a distal opening for admitting fluid flow. The internal lumen is filled with a fluid or other media which can transmit pressure along the guide wire. The pressure measurement system includes a pressure transducer in fluid communication with the internal lumen of the guide wire. Pressure waveform in the body vessel are transmitted through the fluid or media from the distal opening of the internal lumen to the pressure transducer. The pressure transducer is in communication with a processor (with display) for determining the pressure acting on the pressure transducer. A pump with a fluid reservoir in fluid communication with the internal lumen of the guide wire is controlled by the processor. The pump introduces additional fluid into the guide wire lumen.

### BACKGROUND OF THE INVENTION

The present invention relates generally to the field of intravascular guide wires used in the diagnosis and treatment of diseased vasculature, such as stenosis or lesions that can form in a patient's vasculature, and more particularly, to systems and methods which include a lumen based pressure measuring guide wire with a fluid filled lumen and pressure transducer that can measure blood pressure distal to a lesion or stenosis and thus, facilitate the calculation of the Fractional Flow Reserve (“FFR”) for a lesion or stenosis within a body vessel, such as a coronary artery.

It has been determined that measuring FFR for a coronary lesion (a blood flow constriction) can provide a physician with a good measure of how functionally obstructive the lesion is to coronary blood flow and if it is obstructive enough to warrant treatment. FFR is a value calculated from physiological pressures that can identify if a narrowing in the artery is causing significant blood flow reductions and can guide an interventional cardiologist to decide if a lesion (narrowing) requires an intervention (such as angioplasty and stenting). Additionally, once an intervention has been performed, the FFR value can guide the interventional cardiologist to decide if the intervention has been sufficiently successful or if further intervention (increase in artery ID) is warranted. Currently, physicians generally utilize angiography techniques to determine which artery locations have been narrowed and the extent to which blood flow has been obstructed. FFR measurement is meant to be an adjunct to angiography. Clinical studies have shown that making the treatment decision based on the FFR measurement value saves both money (fewer interventional catheters used and less procedure time) and improves patient outcomes relative to decisions that rely solely on angiography.

FFR can be calculated using hyperemic blood pressure measurements at locations distal and proximal (usually the Aortic or systemic blood pressure) of the lesion. Devices that are used today for this purpose may include a guide wire-like member with some type of pressure sensing device incorporated into the device to make the distal pressure measurement. Such devices are often referred to as pressure sensing guide wires since they may provide the dual function of acting both as a conventional guide wire and a pressure measuring device. In some FFR calculation techniques, the proximal pressure measurement can be obtained from a conventional systemic arterial BP method or from a pressure transducer attached to the guide catheter used to access the coronary artery. In other FFR calculation techniques, the pressure sensing guide wire is withdrawn to measure the proximal pressure.

Currently, commercial pressure sensing guide wires include a pressure sensor mounted near the distal tip of a hollow guide wire which requires one or more jacketed leads (electrical or optical) from the pressure sensor to be run though the lumen of the guide wire and back to the proximal end of the guide wire where the pressure sensor's output signal can be accessed to obtain the pressure measurements needed to calculate FFR. One problem associated with such pressure sensing guide wires can be the costs of manufacturing such a device. Part of the high cost can be attributed to the expense of the extremely small and sensitive pressure sensor needed to be placed at the distal end of the guide wire, along with the labor costs required to mount the sensor and run the electrical or optical leads through the length of the guide wire. Another problem associated with such pressure sensing guide wires relates to the fact that the small pressure sensor mounted on the guide wire may not be as accurate or as stable as a standard blood pressure sensor (for design/space saving reasons), requiring frequent calibration or drift correction procedures. Also, since these devices have electrical or optical leads running the length of the guide wire, it is important to maintain a properly sealed connection between the leads and the pressure sensor to prevent surrounding fluid from entering the connection. Even tiny breaches of liquid into the connections may cause errors in obtaining an accurate pressure measurement.

Additionally, current commercial pressure sensing guide wires may not provide the desired range of performance associated with conventional guide wires. Because these types of pressure sensing guide wires incorporate a pressure sensor along with a lead lumen of significant OD relative to the typical 0.014″ OD guide wire, the ability to design a pressure sensing guide wire with a range of distal end designs and performance variations is somewhat compromised. Accordingly, currently available pressure sensing guide wires do not provide the interventional cardiologist with the needed and expected range of performance and design choices that conventional guide wires provide. Such pressure sensing guide wires often have problems accessing challenging lesions and also may have problems delivering an interventional catheter to the lesion, if the FFR measurement shows that treatment is needed. Thus, in some lesions, FFR measurements cannot be performed because the pressure sensing guide wire is not capable of being positioned distal to a tight lesion in tortuous anatomy. Additionally, in such a scenario, the pressure sensing guide wire may have to be replaced with a conventional (front line) guide wire in order to deliver the interventional catheter to the treatment site. Currently, it is common practice for many physicians to not attempt to use the pressure sensing guide wire to deliver the interventional catheter to the treatment site, due to support and tracking issues with the pressure sensing guidewire.

Other FFR pressure sensing guide wires have been proposed which utilize a guide wire having a single fluid filled lumen extending from its distal to proximal end, with a pressure transducer attached near the proximal end of the guide wire. Such a pressure sensing guide wire may solve some of the problems associated with the pressure sensing guide wires mentioned above that incorporate the pressure sensor at the distal end of the guide wire. For example, long electrical/optical leads can be eliminated and lower cost and more stable pressure transducers could be used at the proximal end of the guide wire. Such design changes should decrease labor and production costs. However, measuring pressure through a fluid filled tubing forming the guide wire may create unwanted distortions/dampening in the pressure waveform. Such distortions create measurement errors since the pressure being exerted on one end of the guide wire lumen may no longer be equal to the pressure measured on the other end of the guide wire lumen. Accordingly, in order to more accurately measure pressure through such fluid filled guide wires, these unwanted distortions must be corrected.

FIG. 1 shows a diagrammatic illustration of an embodiment of a particular fluid filled lumen based pressure sensing guide wire system. The various components forming the system of FIG. 1 are clearly marked. The theory and operation of conventional blood pressure measurement systems (including a pressure measuring guide wire that incorporates an optical pressure sensor) are known and are described in “BLOOD PRESSURE AND SOUND” by Robert A. Peura. Current conventional/commercial pressure measuring guide wires incorporate a solid state/hybrid circuit pressure sensor.

Generally, blood pressure is a changing pressure with a minimum pressure which is above 0 and with a fundamental frequency tied to the heart rate (expressed as Beats Per Minute “BPM”). A blood pressure measurement system is usually expected to accurately measure blood pressures at heart rates from about 50 to 120 BPM. In order to accurately measure blood pressure, the measurement system should have a frequency response that is about ten times the heart rate. Thus, a frequency response of 0-20 Hz (Hertz, cycles per second) is usually required for a blood pressure measurement system. For a measurement system whose frequency response can be characterized by a time constant (t_{c}), the upper limit of the low pass frequency response (the cut off frequency f_{c}) can be expressed as:

$${f}_{c} = \frac{1}{2 {t}_{c} π} \qquad EQN 1$$

Thus, in a standard pressure measurement system whose frequency response can be characterized by a time constant (t_{c}) and requires a cut off frequency (or frequency response) of 20 Hz or greater, the time constant (t_{c}) must be 0.008 seconds or less. As will be explained in greater detail below, such a short time constant particularly poses a grave problem for pressure sensing guide wire systems which utilize fluid filled lumens since the required minimum time constant will be difficult to achieve in order to accurately measure the blood pressure at the distal end of the guide wire lumen.

Accordingly, pressure sensing guide wires which utilized a fluid filled lumen and a pressure transducer in fluid communication with the proximal end of the lumen were faced with the following problems:
1. As is stated above, the required time constant of the system is so low that larger ID guide wire lumens are needed in order to accurately measure pressure. To make accurate pressure measurements, the lumen ID had to be even larger than those in current commercial pressure sensing guide wires which utilize pressure sensors at the distal tip. Thus, the ability of this guide wire to access lesions and deliver interventional catheters was far inferior.
2. During pressure measurement and insertion into the body, a significant volume of blood entered and left the distal end of the guide wire lumen with each heartbeat. This rapidly washed away any heparin (anticoagulant) in the initial flushing solution from the distal exit of the guide wire lumen and allowed blood clots to rapidly form in the region of the distal lumen opening, resulting in clogging of the lumen and a loss in pressure sensing ability. The guide wire lumen thus required frequent flushing to remove clots. Even small clots that did not completely close any part of the lumen would degrade the quality (frequency response/accuracy) of the pressure measurements.
3. The system had to be thoroughly flushed of air bubbles. Even small air bubbles would degrade the quality (frequency response/accuracy) of the pressure readings. The removal of stubborn air bubbles during the numerous flushing procedures needed to remove clots could be quite difficult and annoying.

The root cause of these problems can be attributable to the inherently high (RC) time constant (low frequency response) of the pressure measurement system. Only under relatively ideal conditions could the time constant of the system be kept low enough to accurately measure blood pressure. Generally, the fluid mass and the amplitude and rate of change of the fluid flow rates in a lumen based guide wire pressure measurement system are sufficiently small that inertial effects are virtually negligible. Practical pressure transducers and connection components making up the measurement system at the proximal end of the fluid filled guide wire lumen have a certain compliance (C) value, which is difficult and expensive to minimize further. Because of the need to have a long and small ID lumen in a functioning pressure sensing guide wire with an OD of only 0.014″, the guidewire lumen has an inherently high fluid flow resistance (R). The product of this system flow resistance (R) and the system compliance (C), namely, the (RC) time constant of the system, describes the frequency response of this conventional pressure measurement system. If the (RC) time constant is too high, then the pressure measurement system cannot respond fast enough to blood pressure changes at the distal end of the guide wire lumen and thus, cannot provide an accurate blood pressure measurement. If the guide wire lumen ID is too small, or if a clot in the lumen or at the distal end of the lumen raises the fluid flow resistance (R), the already high (RC) time constant (near the 0.008 second limit) increases, making it virtually impossible to accurately measure blood pressure. Additionally, any air bubbles trapped/present in the guide wire lumen, pressure transducer or fluid connection system raises the system compliance (C), also raising the already high (RC) time constant, making it virtually impossible to accurately measure blood pressure.

The physical effects of this phenomenon in prior art lumen based guide wire pressure measurement systems can be explained as follows. Compliance (C) is the property of a (compliant) system to increase its volume in response to an increase in pressure. Accordingly, the higher the compliance, the more of a volume change is required for any given pressure change. An air bubble trapped/present in the measurement system will increase the overall system compliance (C) because the volume of air changes considerably with a change in pressure (compared to a fluid, the connection components and the pressure transducer). As the pressure of the blood increases, the fluid in the guide wire lumen flows into the pressure transducer and the connection components and the pressure of the fluid in contact with the pressure transducer and the connection components rises. Without the pressure of this fluid rising to that of the blood pressure or very near to blood pressure, the pressure transducer cannot accurately sense the pressure of the blood. As the pressure of the blood decreases, the fluid in the pressure transducer and its connection components (the compliant portion of the measurement system) flows out (into the guide wire lumen) causing the pressure of the fluid in contact with these components to also drop. Without the pressure of this fluid dropping to, or very close to, that of the actual blood pressure, the pressure transducer of the measurement system cannot accurately sense the pressure of the blood. If the guide wire lumen ID is too small or becomes clotted, then, as is mentioned above, the flow resistance (R) of the lumen increases. If the flow resistance (R) becomes too high, then fluid flow rate within the guide wire lumen may not be large enough for enough fluid to flow into or out of the compliance of the measurement system portion that contains the pressure transducer such that the pressure in that compliant portion can increase or decrease to the value or near enough to the value of the actual blood pressure and thus, allow the pressure transducer to sense/measure the true blood pressure and provide an accurate pressure measurement as the blood pressure changes. If the compliance (C) of the system becomes too high, for instance, due to the presence of air bubbles, the volume of fluid required to raise or lower the pressure within the transducer and connection components to closely follow the true blood pressure may be too high to allow the existing guide wire lumen flow rate to be adequate. Moreover, even in the system's ideal operating state, the resulting constant inflow and outflow of fluid from the guide wire lumen can quickly wash away any anticoagulant present in flushing fluid at the distal opening of guide wire lumen, which results in rapid clotting, which causes the guide wire lumen flow resistance (R) to increase (guide wire lumen flow rates to decrease) and thus, for the measurement system's ability to follow blood pressure changes to be significantly degraded, as previously described.

The problems associated with the conventional system depicted in FIG. 1 can be further explained. When the guide wire lumen is flushed, air bubbles that are introduced or remain at the end of the flush may become trapped, for instance, in the portion of the stopcock attached to the proximal connection and/or trapped in the proximal region of the guide wire lumen. These air bubbles will remain in the system during pressure measurements and will thus increase the system's overall compliance (C). As a result, the system's overall (RC) time constant will increase and cause the system's frequency response to be lowered. Air bubbles can be generated and introduced into the system during any flush of the system. Since the formation of blood clots at the distal end of the guide wire lumen is also common and also causes the system's frequency response to be degraded, flushing to attempt remove these clots is also common. Thus, due to these occurrences, the development and manufacturing of a guide wire lumen based pressure measurement system that can accurately measure blood pressure in a patient's vasculature is quite challenging and can't compete with pressure sensing guide wires that mount pressure sensors in their distal portions.

What is needed then is a pressure sensing guide wire technology that does not compromise the performance of the guide wire while still providing accurate blood pressure measurements. Guide wire pressure sensing systems are needed that allow pressure sensing guide wire design variation and performance much nearer to that of conventional guide wires. Such a guide wire pressure sensing system would allow FFR calculations to be obtained more reliably with fewer guide wires being used in the procedure, which will reduce the overall procedure time and cost due to the elimination of the need to exchange guide wires. It would also be beneficial if a smaller guide wire lumen ID can be utilized than is needed for a conventional fluid filled lumen guide wire system or is needed for the electrical or optical lead or leads for a conventional pressure sensing guide wire that mounts a distal pressure transducer. The elimination of the placement of a pressure sensor at the distal end of the guide wire lumen also could allow for greater distal end design options and allow pressure sensing guide wire clinical performance to be considerably improved, resulting in time and cost savings while improving patient outcomes. The present inventions satisfy these and other needs.

### SUMMARY OF THE INVENTION

The present invention is directed to a medical system which utilizes a fluid filled guide wire lumen and a unique pressure measurement system that accurately measures blood pressure in a vessel. The present invention utilizes a guide wire having a distal end, a proximal end, an internal lumen having a distal opening to allow flow out of the internal lumen and a proximal opening for admitting fluid flow into the internal lumen. The internal lumen can be filled with a fluid or other liquid media which can flow in the lumen of the guide wire. The proximal opening of internal lumen of the guide wire is in fluid communication with a pressure measurement system which includes a pressure transducer and other compliant components. These fluid filled compliant components of the measurement system are injected with a volume of fluid that raises the pressure of the fluid in the measurement system to a pressure that exceeds the blood pressure. This causes fluid to flow out of the measurement system (into the proximal opening of guide wire lumen, down the length of the guidewire and out of the distal opening of the guide wire lumen) and for the pressure in the measurement system to slowly fall (at rate determined by the system's relatively long RC time constant). The pressure being exerted on the pressure transducer by the fluid or other liquid media within the compliant components can thus, always be accurately measured by the measurement system. The blood pressure to be measured (calculated) is the pressure at the distal opening of the guidewire lumen. If the blood pressure increases, the pressure difference across the flow resistance of the guide wire lumen decreases, thus the flow rate into the proximal end of the guidewire decreases and thus, the rate of the fall in pressure in the measurement system decreases. If the blood pressure decreases, the pressure difference across the flow resistance of the guide wire lumen increases, thus the flow rate into the proximal end of the guidewire increases and thus, the rate of the fall in pressure in the measurement system increases. The rate of fall (decay) of the pressure of the fluid in the measurement system is determined by the RC time constant of the measurement system and the pressure at the distal opening of the guide wire lumen. Measuring the rate of pressure decay at a known (usually a very low or zero) pressure, allows for the determination (calculation) of the value of the RC time constant of the measurement system. With the RC time constant of the measurement system known, the pressure at the distal opening of the guide wire lumen may be determined (calculated). The pressure transducer is in communication with a processor (with display) for determining the pressure acting on the pressure transducer and for calculating the RC time constant(s) of the measurement system and then the pressure at the distal opening of the guide wire lumen (the blood pressure). A pump with a fluid reservoir may be in or may be put into fluid communication with the compliant portion of the measurement system, as determined and controlled by the processor. The fluid pressure acting on the pressure transducer is communicated to the processor, which is programmed, at least, with an algorithm to either calculate the RC time constant(s) or the blood pressure and to activate the pump when the pressure falls to or below a predetermined value (or a predetermined time has elapsed) and to deactivate the pump when the pressure rises to or above a predetermined value. The pump is configured/connected to cause fluid or other liquid media to flow into the compliant portion of the measurement system according to the algorithm. The constant flow of fluid or other liquid media into the internal lumen of the guide wire helps to prevent clotting of blood within the guide wire lumen and at the distal opening of the guide wire lumen. This flow also removes any transitory air bubbles in the measurement system, which could cause variations in the RC time constant of the system and thus, inaccuracies in the determined (calculated, measured) blood pressure.

Generally, the internal lumen of the guide wire has a particular flow resistance (R) value and the measurement system has a particular compliance (C) value which is expressed as the property of the system to increase its volume in response to an increase in pressure exerted on the system. The product of the flow resistance (R) value and the measurement system compliance (C) value defines a time constant (RC) value for the system, but, as will be discussed later, this time constant doesn't determine the frequency response of the blood pressure measurements in the system of this invention. The various components which form the system may include the tube that connects the pressure transducer to the guide wire (referred to as the “connection tube”), along with other proximal connection tubing, the pressure transducer and a check valve or other valve. All of these components determine the total compliance of the entire system (the guide wire has a compliance value as well, but it is negligible compared to the other components of the system). The total compliance (C) of the system times the flow resistance (R) of the guide wire lumen produces a time constant (RC) for the system which should be large enough to allow the collection of data for, at least, one full blood pressure cycle during the pressure decay of the compliant system attached to the proximal end of the guide wire lumen.

The present invention creates a pressure measurement system which relies on a high compliance or a high (RC) time constant of the compliant portion of the measurement system attached to the proximal end of the guide wire lumen. The compliance of the guide wire lumen itself is negligible because of the high modulus (stiffness) metals that are used to construct a guide wire. As was previously addressed above, a conventional system must have a time constant that is equal to or less than about 0.008 seconds to accurately measure blood pressure (at 120 BPM). The pressure measurement system of the present invention, however, can operate with an (RC) time constant on the order of about one (1) second or greater. With a much greater allowable (RC) time constant value and range (of the compliant portion of the measurement system attached to the proximal end of the guide wire lumen), any captured air bubbles have much less impact on the measurement system of the present invention, provided that no new air bubbles are introduced or existing air bubbles within the system and guide wire lumen are not removed after the (RC) calibration step for system use has been performed. Because the pressure measurement system of the present invention does not require manual flushing (the system can automatically flush itself), any introduction or removal of air bubbles into the system can be easily avoided. The frequency response of the pressure measurement system of the present invention, in turn, is not tied to the (RC) time constant of the measurement system as it is in conventional systems. Instead, the frequency response of the pressure measurement system of the present invention is tied to the (inertial) time constant of the change in flow rate in the guide wire lumen in response to a change in pressure (due to a change in the blood pressure) across the length of the guide wire lumen.

Thus, the present invention may utilize a guide wire having a higher guide wire lumen flow resistance (R) than say a conventional system. Accordingly, the diameter of the guide wire lumen that can be used in accordance with the present invention can be smaller than the lumen diameters used with prior and present conventional systems. Thus, the performance of the guide wire is much less compromised by the presence of lumen, because the ID of the lumen is smaller and thus, has less impact on the mechanical properties of the guide wire's design than prior and present conventional guide wire pressure measuring systems. Additionally, there is no pressure transducer mounted at or near the distal end of the guide wire and thus, the mechanical properties of the guide wire's distal design, the design portion most responsible for its tracking, vessel branch selection, lesion crossing and catheter delivery/support properties, are much less compromised than present conventional guidewire pressure measuring systems. The size of the outer diameter of a guide wire which can be utilized with the present invention can be the same as any typical 0.014″ or larger guide wire.

During pressure measurement, when the measurement system of the present invention is attached to the proximal end of the guide wire, there is always a dilute fluid flow (usually including an anticoagulant such as heparin) down the guide wire lumen, so the anticoagulant in the fluid of the lumen is continuously replaced. When the measurement system is not attached to the guide wire, as may be desired during the initial positioning of the guide wire distal to the lesion, the lumen is filled with an anticoagulant fluid (for example, a highly heparinized saline) and the proximal end of the guide wire may be capped. The guide wire lumen has a very low (negligible) compliance, as discussed above, because a guide wire is made of metal, which has a very high modulus. Accordingly, there is negligible inflow and outflow from the distal end of the guide wire lumen with its proximal end capped. Thus, the anticoagulant must diffuse out of the distal end of the guide wire lumen to be removed/lost. Thus, the guide wire may be clot free much longer than a conventional fluid filled guide wire that is not capped, but remains connected to a compliant system.

Diffusion out of a long lumen of a guide wire can be a very slow process, long enough to allow for guide wire positioning. As the anticoagulant fluid is washed away at the distal end of the lumen, its concentration near the distal end of guide wire lumen drops. The more the anticoagulant concentration drops, the greater the concentration difference between the fluid in distal end of the lumen and in the proximal end of the lumen becomes and thus, the greater will be the diffusion flow of the anticoagulant from the proximal portions of the lumen to the distal portions of the lumen. Additionally, because the volume of the guide wire lumen is so small, the concentration of the anticoagulant in the fluid during guide wire positioning can be very high and not have any appreciable effect on the patient's anticoagulation state. Thus, the distal end of the guide wire lumen of the present invention will remain virtually clot free much longer than the previously described conventional systems. The time required for a sufficient amount of anticoagulant to diffuse out of the distal end of the guide wire lumen such that the anticoagulant concentration is too low to prevent clots can be increased by placing a high concentration anticoagulant fluid into the guide wire lumen just prior to guide wire capping and initial guide wire positioning. Once the guide wire is in position (within the body lumen and usually distal to the lesion), the measurement system of the present invention can then be attached to the proximal end of the guide wire and the constant flow will prevent any blood clotting during the distal FFR pressure measurement and until pressure measurements are no longer required. The measurement system of the present invention can remain attached and clot free while the guide wire is retracted and the proximal FFR pressure measurement is made, if desired, without any significant inconvenience to the physician.

In a conventional system, when the guide wire lumen is flushed, any air bubbles that are introduced at the end of the flush remain, for example, in the portion of the stopcock attached to the proximal connection, in the proximal components of the system or in the guide wire lumen. These air bubbles remain in the system during pressure measurement and thus increase the system's compliance, which increases the system's measurement time constant and lowers its frequency response. The same is true when the pressure transducer is flushed. In the system of the present invention, there is always a pressurized flow through the wetted components forming the measurement system, along with internal lumen of the guide wire, while the measurement system is connected. Thus, if a flush of the guide wire lumen should be required, any bubbles introduced would most likely be washed away and only have a very temporary effect on the system. Additionally, all of the wetted system components are continually and automatically flushed by a closed IV-like system. Widely available IV system components may be incorporated to ensure that air bubbles are not a part of the automated periodic flow into the compliant system.

In another aspects, the present invention is directed to methods for measuring pressure, particularly for calculating the Fractional Flow Reserve of a lesion within a body vessel, utilizing a system made in accordance with the present invention.

These and other advantages of the present invention will become apparent from the following detailed description of preferred embodiments which, taken in conjunction with the drawings, illustrate by way of example the principles of the invention.

### BRIEF DESCRIPTION OF THE DRAWINGS

FIG. 1 is a diagrammatic illustration of a prior art lumen based pressure sensing guide wire system.

FIG. 2 is a diagrammatic illustration of a particular embodiment of a lumen based pressure measurement guide wire system made in accordance with the present invention which can be used to measure blood pressure at the distal end of the guide wire.

FIG. 3 is a side-elevational view showing the distal end of an embodiment of a guide wire which can be used with the present invention.

FIG. 4 is a side-elevational view showing the distal end of another embodiment of a guide wire which can be used with the present invention.

FIG. 5 is a side-elevational view showing the distal end of another embodiment of a guide wire which can be used with the present invention.

FIG. 6 is a side-elevational view showing the distal end of another embodiment of a guide wire which can be used with the present invention.

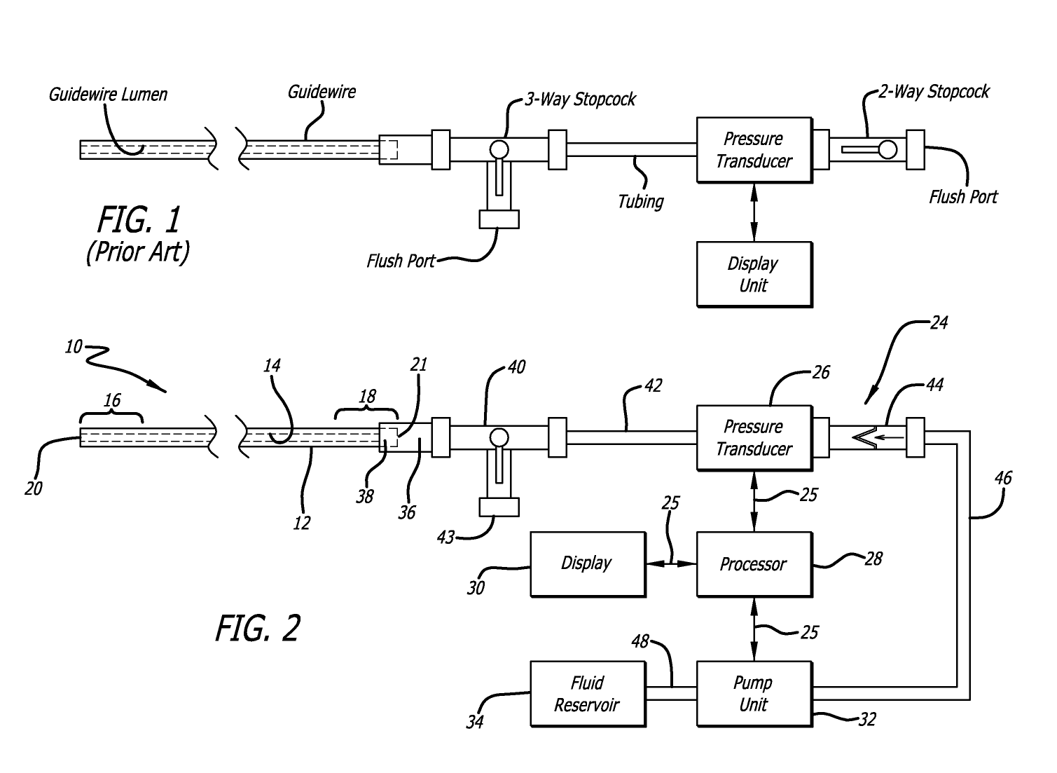

### DETAILED DESCRIPTION OF THE PREFERRED EMBODIMENTS

A particular embodiment of a lumen based pressure measurement guide wire system 10 made in accordance with the present invention is schematically shown in FIG. 2. The system 10 includes a guide wire 12 having an internal lumen 14 which extends from a distal region 16 to a proximal region 18. The internal lumen includes a distal opening 20 and a proximal opening 21. The distal opening 20 is designed to come in contact with blood flowing within a body vessel, such as an artery. The distal opening 20 shown in FIG. 2 extends across the diameter of the guide wire 12 and is just one representation of an opening which could be placed on the guide wire 12. For example, see the distal opening 20 shown in FIG. 6 in which the opening is cut into the sidewall of the guide wire. Many different embodiments of the distal opening 20 location and geometry could be used and this invention is not limited just to the embodiments shown.

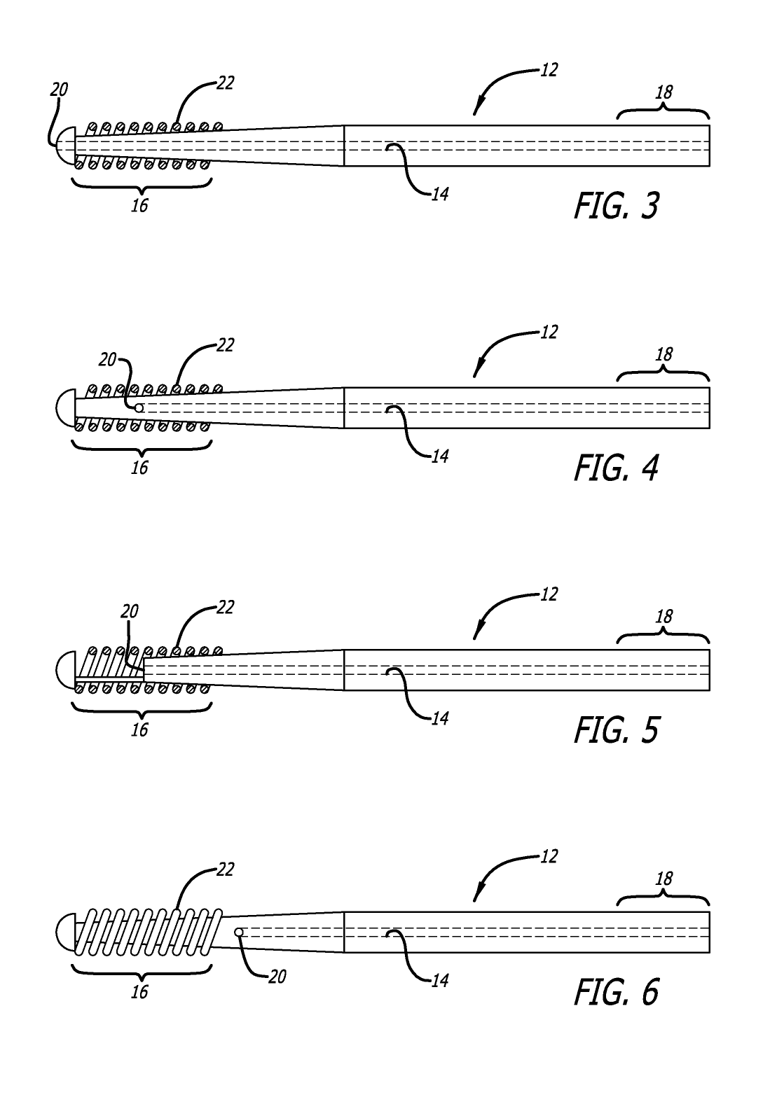

The guide wire 12 may include a flexible tip coil 22 (See FIGS. 3-6) and tapered core flexible working sections that are typical features of the distal portions of a guide wire and are consequently well known and understood in the art. In FIGS. 3-6, the coil tip 22 is shown in section so that the location of the distal opening 20 can be better visualized. The overall diameter of the guide wire 12 can be the typical 0.014 inches for cardiac guide wires; however, the invention is not limited to this dimension. In addition, it is common to keep the outer diameter as constant as practical throughout the length of the guide wire 12. However, the invention is not limited to this requirement and there are embodiments of this invention where the outer diameter of the guide wire changes throughout the length of the guide wire. The internal lumen 14 shown in FIG. 2 is to be filled with a fluid (not shown) or media that can flow through the lumen 14 of the guide wire 12. The fluid or other media should be selected such that the pressure decay time constant and frequency response (discussed later) of the system are maintained at acceptable values.

FIG. 2 shows the guide wire 12 in communication with the pressure measurement system 24 of the present invention which includes a pressure transducer 26 which is in fluid communication with the internal lumen 14 of the guide wire 12. Pressure variations in the body vessel cause the pressure across the guide wire lumen 14 (from the distal opening 20 of the internal lumen 14 to the opening at the proximal end 38 of the guide wire 12) to vary. The pressure varying across the internal lumen 14 of the guide wire 12 causes the flow rate of the fluid out of the measurement system 24 and into the guide wire lumen 14 to vary. Varying the flow rate out of the compliant system of measurement system 24 causes the rate of the pressure decay within the compliant system to vary. This (varying) rate of pressure decay can be accurately sensed by the pressure transducer 26 of the measurement system 24. The processor 28 will use the pressure data sampled from pressure transducer 26 and previously stored RC time constant data (discussed later) to provide a calculated measure of the (varying) blood pressure acting on the fluid at the distal opening 20 of the guide wire 12. In this manner, the pressure of the blood within the body vessel that is in fluid contact with the distal opening 20 of the internal lumen 14 is measured (calculated).

Referring again to FIG. 2, the measurement system 24 further includes a processor 28 (with display 30) for measuring and processing the pressure readings being provided by the pressure transducer 26. The pressure transducer 26 could be electrically or optically in communication with the processor 28 or in communication via radio waves (the connection depicted by arrows 25). The signal from the pressure transducer 26 is sampled and converted by the processor 28 to a pressure or pressure related readings that correspond to the pressure within the compliant system of measurement system 24.

The display 30 provides the user with immediate and previously collected information (from at least one blood pressure cycle) regarding the blood pressure being measured in the patient's body vessel. The display 30 also provides the user with the means to select the pressure data to calculate and store the RC time constant data for later use in calculating blood pressures or to calculate and display the calculated blood pressure. It should be appreciated there are numerous medical instrumentations and data display formats that could be used to process the signals from the transducer 26 and provide the RC time constant storage results and status or the blood pressure information to the user.

A pump 32 with a fluid reservoir 34 is in fluid communication with the proximal opening 21 of the internal lumen 14 of the guide wire 12 and the compliant system portions of measurement system 24 via a check valve 44 (or other valve). The pump 32 is in communication with the processor 28 as the actions of the pump 32 are controlled by the processor 28. Any pressure variations acting on the pressure transducer 26 are communicated to the processor 28 which is programmed with an algorithm to activate and deactivate the pump 32 in response to a low pressure limit and a high pressure limit, respectively. The pump 32 is configured to cause fluid or media to flow into the compliant system portions of the measurement system 24 from the fluid reservoir 34 according to instructions from the processor 28 when the low pressure limit is reached and to continue until the high pressure limit is reached. The low pressure limit is set such that it is always greater than the highest blood pressure. RC time constant or blood pressure calculations may only be produced from pressure data collected as the pressure in the compliant system portions of the measurement system 24 decays from the high pressure limit to the low pressure limit. The time required for the pressure to decay from the high pressure limit to the low pressure limit must be adequate to provide pressure data collections over the interval of one or more blood pressure cycles. Because the low pressure limit is always above the highest blood pressure, there is a continuous flow of fluid or media into the internal lumen 14 of the guide wire 12 that helps to prevent clotting of blood within the guide wire lumen 14 and near its distal opening 20 and tends to eliminate the adverse effects of transitory (not captured) air bubbles found in the guide wire internal lumen 14 and the compliant system components making up the measurement system.

The pressure measurement system 24 may include appropriately chosen commercially available components which provide various functions to the system 10 at a lower cost than custom designed components. Again referring to FIG. 2, the pressure measurement system 24 includes connection tubing which is used to place the various major components of the system in fluid communication with each other. For example, a proximal connection member 36 is shown in contact with the proximal end 38 of the guide wire 12 to establish a mating connection between the internal lumen 14 and a 3-way stopcock 40. This stopcock 40 provides a guide wire flush port 43, which could be used, if needed, to flush fluid into the internal lumen 14 of the guide wire 12 without causing pressure transducer 26 to experience very high pressures, which could degrade its performance. In some embodiments, the 3-way stopcock may be omitted. The 3-way stopcock 40 is, in turn, connected to a connection tubing 42 which provides the conduit for placing the pressure transducer 26 in fluid communication with the internal lumen 14 of the guide wire 12. The other end of the pressure transducer 26 can be connected to a check valve 44 which provides a one-way conduit for advancing fluid from the fluid reservoir 34 into the compliant system portions of the measurement system. Additional connection tubing 46 is used to fluidly connect the check valve 44 to the pump 32. Connection tubing 48, in turn, is used to fluidly connect the fluid reservoir 34 with the pump 32.

In the preferred embodiment of the invention, the connection member 36 is designed as a temporary/removable means for attaching the guide wire 12 to the measurement system 24. Accordingly, this connection member 36 should easily removable or attachable to the guide wire 12 by the user. For instance, connection member 36 may contain a feature much like that of common hemostasis valve designs that seals around the OD of the proximal region 18 of guide wire 12 with a deformable member (like an o-ring) as a result of the linear motion of a screw-like member when rotated by the physician. However, the present invention is not limited to this feature and the connection member 36 could be formed, for example, as an integral component of the guide wire 12, however; this is less preferred because the presence of connection member 36 may interfere with any catheter or other device that the physician may wish to subsequently deliver over guide wire 12. Additionally, the connection member 36 could be formed as an integral portion of the 3-way stopcock 38 without departing from the spirit and scope of the present invention. It should be appreciated that a number of different connection components could be used to fluidly connect the pressure transducer 26 to the internal lumen 14 of the guide wire 12. FIG. 2 shows just one of the many ways to fluidly connecting these two components together.

The processor 28 may provide the operating power (electrical or optical) to the pressure transducer 26. The processor 28 receives pressure information from the pressure transducer 26, performs mathematical calculations on the pressure data and other data, operates the pump 32 and provides a user interface via the display 30. The user (generally a physician) may select the operating and display modes of the measurement system via the display 30. The display of the blood pressure and other calculated data may take the forms normally seen in the hospital setting and/or be adapted to facilitate and/or perform FFR calculations or to facilitate the successful determination of the RC time constant(s) of the compliant system as part of the set-up procedure. The fluid reservoir 34 can be, for example, a standard IV type fluid container that is filled with a heparinized saline solution.

The portion of the check valve 44 that is connected to the pressure transducer 26, the pressure transducer 26 itself, the connection tubing 42, the 3-way stopcock 40 and the proximal connection member 36 are all in relatively unrestricted fluid communication with each other and comprise a portion of the pressure measurement system and can be referred to as the “compliant system” connected to the proximal end of the guide wire lumen. The product of the compliance value (C) of the compliant system and the flow resistance value (R) of the guide wire 12 internal lumen 14 determines the minimum RC time constant of the measurement system. In some embodiments, the compliance of connection tubing 42 is chosen to be a value that provides, in conjunction with the other components of the compliant system, a compliance value that provides the desired minimum RC time constant value of the measurement system. For instance, the compliance of a tube with a constant ID and OD is directly proportional to its length. Thus, adjusting the length of connecting tube 42 will adjust the compliance value of the compliant system. Naturally, as is well known, other design features of a tube may be changed to increase or decrease its compliance. Adjusting the compliance value of the compliant system by changing a feature or features of the connecting tube 42 is often much less expensive than attempting to adjust the compliance values of the other components of the compliant system. The exact position of some of the components in the compliant system relative to each other can be changed. For instance, in FIG. 2, the positions of the pressure transducer 26 and the connection tubing 42 may be reversed. The proximal connection member 36 attached to the guide wire 12 can be, for example, much like a part of a standard hemostasis valve and comprise an o-ring and/or another deformable component that is releasably compressed by a screw-like mechanism of the proximal connection member 36 such that it presses against the OD of the guide wire 12 and forms a seal and a secure leak proof temporary attachment to the proximal end 38 of the guide wire 12. In some embodiments, where the connection member 36 and stopcock 40 are not a single unit, a cap (not shown) could be utilized to seal off the proximal end of connection member 36 after the guide wire lumen 14 is flushed with a high concentration heparinized solution prior to insertion and positioning of the guide wire 12 into the body vessel. This will assure that the guide wire opening 20 and lumen 14 will not become clotted during guide wire insertion and positioning. Attempting to insert and position (move distally) the guide wire while significant portions of the measurement system are attached to its proximal end would be difficult/awkward and, most likely, unsafe. Connection member 36 may be designed to be light weight and streamlined and thus, not materially interfere with the insertion and positioning of the guidewire.

The various functions of the measurement system 24 and the manner in which the system operates to supply fluid to the system 14 will be discussed further. Initially, the system of FIG. 2 is flushed with a fluid or other media that fills the internal lumen 14 of the guide wire 12, along with the various connection tubing 42, 46 and 48 so that a closed fluid system is achieved. When the processor 28 detects that the pressure in the compliant system has dropped below a preset lower limit pressure (or after a predetermined time has elapsed), it turns on the pump 32. The pump 32, in turn, starts to infuse fluid from the fluid reservoir 34 through the check valve 44 and into the compliant system via the connection tubing 46 and 48 until the processor 28 detects that the pressure has reached a preset upper limit pressure. This operating function can also be used to automatically flush the system during the initial set-up. When the preset upper limit pressure in the compliant system is detected, the pump 32 will then be turned off and, in a suitably designed system, the pressure within the connection tubing 46 and 48 drops rapidly to below the low pressure limit in the compliant system and the check valve 44 closes, such that the compliant system is no longer in fluid communication with the pump 32 and connection tubing 46 and 48. The complaint system remains in fluid communication with the guide wire lumen 14 and thus, the fluid flows out of the compliant system into the guide wire lumen and out of the distal opening 20 of the guide wire lumen 14. In some embodiments, the check valve 44 can be replaced by a conventional valve that is opened and closed at appropriate times by the measurement system. In some embodiments, the check valve 44, tube 46 and the pump unit 32 are replaced by a syringe and a syringe pump and thus, the syringe becomes a part of the compliant system. Many component choices and configurations are possible that will provide a suitably compliant system in fluid communication with the guide wire lumen, provide the means to measure the pressure in the compliant system and provide the means to cause the compliant system to attain an internal/fluid pressure equal to a high pressure limit once a low pressure limit is reached (or a predetermined time has elapsed) and thus, provide the means for the method of this invention's pressure measurement to function. In some embodiments, the lower pressure limit is a failsafe limit and the pump 32 remains off for a preset (or calculated) time duration which is sufficient to measure the desired number of blood pressure waveforms (cycles).

The rate at which the fluid flows out of the compliant system is determined by the pressure inside the compliant system, the compliance (C) value of the compliant system, the pressure at the distal opening 20 of the guide wire lumen 14 and the flow resistance value (R) of the guide wire lumen 14. The processor 28 samples the pressure data at known time intervals and computes/displays the pressure at the distal opening 20 of the guide wire lumen 14 from the sampled pressure data and the stored time constant data of the compliant system. The pressure at the distal opening 20 of the guide wire lumen 14 is the blood pressure when the distal opening 20 is positioned in a body vessel such as the coronary artery.

In a practical system, the design of connection tubing 42 may be chosen such that the total compliance of the compliant system causes the time constant of its pressure decay rate to be large enough for the processor 28 to have sufficient time to sample the desired number of blood pressure waveforms (blood pressure cycles or cardiac cycles) before the low pressure limit is reached. When the low pressure limit is reached (or the data sampling time has expired), the computation of the blood pressure or RC time constant (or the storage of pressure data) is terminated and the cycle repeats until blood pressure measurement is no longer required by the user or flushing of the system is no longer desired. It is preferred that once the guide wire 12 has been positioned distal to a lesion, that the measurement system 24 be immediately connected to the proximal end 38 of the guide wire 12 and the pumping cycle immediately initiated to assure that the guide wire lumen 14 does not clot and to “exercise” the compliant system (discussed later). In some embodiments, a cap is removed from the connection member 36 and then the system 24 is attached to the proximal end of the connection member 36.

Generally, the lumen of any guide wire has a particular flow resistance (R) value based on the viscosity of the fluid, inner diameter of the lumen and the length of the guide wire. In the present invention, the compliant system of the measurement system 24 has a particular compliance (C) value which is expressed as the property of the system to increase its volume in response to an increase in pressure exerted on the system. The product of the flow resistance (R) value of the guide wire lumen and fluid used with the present invention and the compliant system's compliance (C) value defines a time constant (RC) value that describes the maximum decay rate of the pressure in the compliant system. In one particular aspect of a system made in accordance with the present invention, each of the various components, which form the compliant system, has a compliance (C) value which can be added together to produce an overall compliance (C) value for the compliant system. The total compliance (C) value of the compliant system times the flow resistance (R) of the guide wire lumen 14 produces a time constant (RC) large enough to allow the collection of data for at least one blood pressure cycle during the pressure decay of the compliant system, which is attached to the proximal end 38 of the guide wire lumen 14. The present invention creates a pressure measurement system that relies on a high (RC) time constant of the guide wire and compliant system portions of the measurement system 24 attached to the proximal end 38 of the guide wire lumen 14. The guide wire lumen 14 also has a compliance (C) value, however, it is negligible compared to the compliance value of the compliant system because high modulus (stiffness) metal is used to construct the guide wire 12.

As was previously addressed above, a conventional guide wire fluid filled lumen based system must have a (RC) time constant that is less than about 0.008 seconds to accurately measure blood pressure (at 120 BPM). The guide wire lumen and compliant system of the pressure measurement system 24 of the present invention, however, can operate with an (RC) time constant on the order of about one (1) second or greater. With a much greater allowable (RC) time constant value and range of values, any captured air bubbles have much less impact on the measurement system 24 of the present invention compared to prior art, provided that no new air bubbles are introduced or existing air bubbles within the compliant system and guide wire lumen 14 are not removed after the (RC) calibration step for system use has been performed. Because the pressure measurement system 24 of the present invention does not require manual flushing (the system can automatically flush itself), any introduction or removal of air bubbles into the system 24 can be easily avoided. Moreover, the frequency response of the pressure measurement system 24 of the present invention, in turn, is not tied to the (RC) time constant of the compliant system attached to the proximal end of the guide wire lumen as it is in prior art conventional systems. Instead, the frequency response of the pressure measurement system 24 of the present invention is tied to the (inertial) time constant of the change in flow rate in the guide wire lumen 14 in response to a change in pressure (the blood pressure) across the length of the guide wire lumen 14.

As a result of the high (RC) time constant that is utilized, the present invention may utilize a guide wire having a higher guide wire lumen flow resistance (R) than say a conventional/prior art system. Accordingly, the diameter of the guide wire lumen 14 can be smaller than the lumen diameters used with prior and present conventional guidewire pressure measuring systems. Thus, the performance of the guide wire is much less compromised by the presence of an ID, because the ID is smaller and thus has less impact on the mechanical properties of the guide wire's design than prior and present conventional guide wire pressure measuring systems. Additionally, there is no pressure transducer mounted at the distal end of the guide wire and thus, the mechanical properties of the guide wire's distal design, the design portion most responsible for its tracking, vessel branch selection, lesion crossing and catheter delivery/support properties, are much less compromised than prior or present conventional guidewire pressure measuring systems.

Most or all of the compliant system components (and other wetted components of the system) may be pre-assembled and also may comprise a housing (not shown) that ensures that the connection tubing 42 is not bent or otherwise mechanically disturbed during pressure measurement use. This prevents introducing pressure changes into the compliant system unrelated to the blood pressure (prevents the inadvertent introduction of noise into the measurement system 24). Also, a pre-assembled compliant system may have its components bonded to together in a manner that precludes system leaks, which could also cause pressure measurement problems. A preassembled unit or a subassembly of the pre-assembled unit (during the manufacturing process) may have its pressure transducer 26 calibrated using air pressure and thus, it may be easily tested for leaks in the pre-assembled unit by closing it off from the pressure source and observing the pressure decay rate of air in the pre-assembled unit using the signal being generated by the pressure transducer 26. A calibration of the pressure transducer 26 may result in calibration constants (or calibration constant related codes) that must be entered into the processor 28 via display 30 for the pressure transducer signal to be accurately processed into pressure data. This pressure transducer calibration information may be directly dialed/entered into the processor 28 by the user via the display 30 or read by the processor 28 using other well-known methods, such as RFID chips, barcodes and the like, which may be incorporated directly into or on the pressure transducer 26 or its cabling or the pre-assembled unit.

In some embodiments, all or most of the fluid-wetted components are provided to the user as a disposable pre-assembled unit and in practical systems, the processor 28, display 30 and pump 32 are reusable equipment. In some embodiments, the active portions of the pressure transducer 26 are re-usable and removably attach into the compliant system to allow the use a high quality transducer system to be economically feasible. In the disclosed embodiment of FIG. 2, the processor 28, display 30 and pump 32 are separate units. However, it is possible to combine these instrumentations into a single unit. In alternative embodiments, the processor 28 and pressure transducer 26 communicate wirelessly. The pump 32 is shown attached to the fluid reservoir 34 via a connection tubing 48. It should be appreciated that the fluid reservoir 34 could be incorporated directly with the pump 32 in order to eliminate the connection tubing 48.

In practical modern systems, pressure and other data are digitally sampled, stored and processed and thus, the following equations 2 and 3 have been derived to be compatible with digital systems. It should be understood that the derivation of related analog results/equations using the teachings of this invention and well known mathematical techniques are trivial to those skilled in the art. The two equations below (EQN 2+3) may be used to calculate the pressure at the distal opening 20 of the guide wire lumen 14 and for calculating the RC time constants of the compliant system. The blood pressure may be calculated using the below equation with the RC time constants of the compliant system, the pressure change in the compliant system and sample time interval data.

Symbols:

R=Flow resistance of the guide wire lumen to the fluid flow

Cₓ=Compliance of the (compliant) system (at the proximal end of the guide wire lumen) during sample time interval (tₓ₊₁−tₓ)

Pₓ=The measured pressure in the (compliant) system at time tₓ

P_{d}=Average pressure (blood pressure) at the distal end of the guide wire lumen within a small sample time interval (tₓ₊₁−tₓ)

tₓ=Sequential sample time measurement number x after the upper pressure limit is attained/fluid injection into the compliant system is stopped

$${P}_{d} = \frac{{P}_{x} + {P}_{x + 1}}{2} - \frac{{RC}_{x} ( {P}_{x} - {P}_{x - 1} )}{{t}_{x + 1} - {t}_{x}} \qquad EQN 2$$

Thus, as long as the value of RCₓ has been determined and stored, the digital equipment will have all the data needed to calculate the average pressure at the distal opening 20 of the guide wire lumen 14 during the small sample time interval tₓ₊₁−tₓ. Because small changes in pressure must be detected, the linearity and resolution of the pressure transducer 26 are important system specifications, especially between the upper and lower pressure limits. With modern electronics, such as sample and hold circuits, operational amplifiers, A/D converters, active filters and the like, converting even small signal changes like (Pₓ−P−ₓ₊₁) into a digital value with a significant number of digits is not very challenging and thus, accurate blood pressures may be calculated. However, it should be noted that the greatest changes in measured pressures will occur right after the upper pressure limit is reached and no more fluid is being pumped into the compliant system. Thus, once the initial transient vibrations due the fluid injection and/or valve closure have died down, the sequential pressure data pairs with the greatest change in pressure may be collected and thus, the most accurate blood pressures calculated.

The value of RCₓ of the system may be calculated and stored by the processor 28 in a calibration step at a time just prior to use (prior to guide wire insertion into the body/vessel). This value can be calculated from compliant system pressure data samples and their sample time interval, provided the distal opening 20 of the guide wire lumen 14 is not yet inserted into the body/vessel and is kept at 0 pressure (or, more correctly, open to the air or submerged a short distance in a wetting bath). The equation for calculating RCₓ is:

$${RC}_{x} = \frac{{t}_{x + 1} - {t}_{x}}{ln ( \frac{{P}_{x}}{{P}_{x + 1}} )} \qquad EQN 3$$

The value of R is determined by the viscosity of the fluid and the radius and length of the guide wire lumen, see EQN 5 below. Thus, for any particular guide wire pressure measurement system design (one set of devices), the value of R is very constant and predictable. In practical systems, the compliance C value is determined by the physical deformation of plastic components (and of any air bubbles) in the compliant system. Plastics are generally not perfectly elastic and under pressure tend to deform over time. Fortunately, at practical pressures and component lumen diameters, this deformation is very slow, but it does have a time related effect on the compliance Cₓ value. However, experiments with commonly available commercial components indicate their compliance Cₓ value stabilizes after a few pump cycles (after the compliant system is “exercised”) and that the most accurate pressure measurements may be made using values of RCₓ that are calculated, processed (filtered/curve fit) and stored for each sample time interval after the upper pressure limit is attained and the compliant system has been exercised.

When the RCₓ values are recorded, the lumen's distal opening is exposed to the atmosphere (atmospheric pressure) or very near the ambient pressure of the atmosphere/submerged a short distance in the guide wire wetting bath. This pressure is considered a zero (0) pressure and the flow rate in the lumen will be a certain value. Blood pressures (and almost all pressures) are measured as the pressures that exceed the ambient atmospheric pressure. When the lumen's distal opening is exposed to the blood in the artery for a pressure measurement, the pressure at the opening is much greater than zero (0) and the flow rate in the lumen will be lower than during the calibration. Thus, pressure in the compliant system will decay faster (reach lower values in a shorter time) during the calibration than during pressure measurements. Thus, during calibration, the time interval between when the pump is turned off/the initial transient vibrations due the fluid injection and/or valve closure have died down and when the low pressure limit is reached will be shorter than during a pressure measurement. Thus, the RCₓ values can only be calculated/stored during a time interval that is always shorter than the time interval during pressure measurement until the low pressure limit is reached. Thus, during pressure measurement, the system will run out of RC values to use to calculate/measure blood pressure before the low pressure limit is reached. Thus, during a pressure measurement, there will always be a time interval before the low pressure limit is reached in which calculation/measurement of the blood pressure cannot be made because there are no more RCₓ values to use in the calculations. To avoid this no pressure measurements possible time interval (to speed up the pressure measurements), when the end of the recorded RCₓ data is reached, pressure measurements can be immediately discontinued. The pump can then be turned on to start the next pressure measurement cycle. During the calibration process, the RCₓ values are recorded and the time interval over which they were recorded are known. Thus, the largest time that pressure measurements (and not run out of RC values) can be taken can be determined.

The RC value is best calculated/recorded not as a constant (i.e. a single value), but as a value that changes with time (“x”, the time after the high pressure limit is achieved/the pump is turned off), as a series of values RCₓ, where RC is a function of time “x”. This is because the practical/low cost (plastic) materials for the measurement system have mechanical properties that change with time when they are under pressure and thus, what for other materials is an RC time constant (the RC value is a constant) is a time variable for a plastic material (i.e. the RC value changes with time).

Because the blood pressure is calculated from the change in pressure decay rate in the complaint system and this change in pressure decay rate is proportional to the change in flow rate in the guide wire lumen 14, the frequency response of the present invention is the same as the frequency response of the flow rate in the guide wire lumen 14 to pressure changes across the length of the guide wire lumen 14. This flow rate frequency response is inertial in nature and also a time constant/low pass filter type of response. The derived equation for calculating the cut off frequency of the frequency response of the flow rate is:

More Symbols:

r=Radius of the guide wire lumen

ρ=Density of the fluid in the guide wire lumen

μ=Viscosity of the fluid in the guide wire lumen

f_{c}=Cut off frequency of the measurement system's response

$${f}_{c} = \frac{4 μ}{{r}^{2} ρ π} \qquad EQN 4$$

### Example of a Pressure Sensing Guide Wire System

For illustration purposes, the frequency response of a hypothetical guide wire lumen will be calculated:

μ=the viscosity of water=8.90×10⁻³ dyne-sec/cm² [gram/(cm-sec)]

p=the density of water=1.0 gram/cm³

r=the radius of the hypothetical guide wire lumen=0.0025 inch=6.35×10⁻³ cm

$${f}_{c} = \frac{( 4 ) ( 8.90 \times {10}^{- 3} )}{{( 6.35 \times {10}^{- 3} )}^{2} ( 1.0 ) ( 3.1416 )} = 281$$

The frequency response of this guide wire example is 0 to 281 Hz, which is much more than sufficient to accurately measure blood pressure. Because the viscosity and density of the fluid (water) will be very close to that of heparinized saline and other suitable anticoagulant solutions and the radius of the guide wire lumen is well within tube drawing capabilities, this frequency result illustrates that the present invention can be designed to have an adequate frequency response to accurately measure blood pressure. This is especially true when one notes that the cut off frequency gets larger with a reduction in the radius of the guide wire lumen 14.

A potential problem of the present invention is the volume and/or rate of fluid flow that can be expected to flow out of the guide wire lumen and into the bloodstream. For illustration purposes, the average flow rate of this flow will be estimated for the hypothetical system of the present invention.

More Symbols:

Q=Average flow rate of the fluid flowing in the guide wire lumen

L=Length of the guide wire lumen

P=Average pressure applied across the guide wire lumen

$$\begin{matrix}R = \frac{8 μ L}{π {r}^{4}} & EQN 5 \\ Q = \frac{P}{R} & EQN 6\end{matrix}$$

The length of an RX guide wire is about 190 cm, so L is set at 190 cm in the hypothetical system. Then R is:

$$R = \frac{( 8 ) ( 8.90 \times {10}^{- 3} ) ( 190 )}{( 3.1416 ) {( 6.35 \times {10}^{- 3} )}^{4}} = 2.65 \times {10}^{9}$$

In the hypothetical system, we will assume that the average pressure (P) is one atmosphere or 1.013×10⁶ dynes per cm². Thus, the average flow rate is:

$$Q = \frac{1.013 \times {10}^{6}}{2.65 \times {10}^{9}} = 3.82 \times {10}^{- 4}$$

The estimated flow rate of 3.82×10⁻⁴ cc per second is a very low flow rate. At that flow rate, it would take about 43.6 minutes to inject 1 cc of fluid into the bloodstream. Thus, the volume of fluid injected into the bloodstream will not be a significant patient safety issue. However, this calculation does show that the pump 32 must be capable of injecting a very small amount of fluid/inject fluid at a very low flow rate into the compliant system in order to control the rate of pressure increase/control the upper pressure limit in the compliant system when the pump 32 is on. It also shows that the check valve 44 must close very well and not leak and all the components of the complaint system cannot be allowed to leak as well. Additionally, it indicates that the volume of the fluid reservoir 34 need not be large, which indicates that a syringe, for instance, with a volume in the 2-5 cc range, and a syringe pump could function as the fluid reservoir 34 and pump unit 32, be a part of the compliant system and eliminate the need for check valve 44 in some embodiments of practical systems, as previously discussed.

In an alternative embodiment, the manual check valve 44 may be replaced with a more conventional valve, one that is controlled by the processor 28. Such a valve control mechanism may be reusable and would be attached to the valve portion of the pre-assembled tubing set, likely in/on the housing (not shown). While the pump 32 is pumping fluid, the controlled valve would remain open and when the upper pressure limit is reached the valve would shut and the pump 32 would be turned off. Experiments also indicate that a syringe pump using a 1 cc syringe may function well as the wetted portion of the pump 32 (a system to refill the syringe from the reservoir is needed), provided the compliance of the connection tubing 46 to check valve/controlled valve (from the pump 32) has a low compliance value and the pressure in tubing 46 is relieved (drops to a low pressure) after the upper pressure limit in the compliant system is attained (for instance, the plunger of the 1 cc syringe may be slightly withdrawn). The higher the compliance value of the connection tubing 46, the greater the flow volume that is required to cause the components on the proximal side of the check valve to reach or exceed the upper pressure limit. Experiments also show that the check valve/controlled valve could be eliminated by connecting a suitability low compliance syringe pump directly to the compliant system. However, this direct connection may not be convenient to the user (syringe pumps are generally heavy and bulky), unless the syringe pump is especially designed for this use.

The present invention has been described in connection with certain embodiments, combinations, configurations and relative dimensions. It is to be understood, however, that the description given herein has been given for the purpose of explaining and illustrating the invention and are not intended to limit the scope of the invention. In addition, it is clear than an almost infinite number of minor variations to the form and function of the disclosed invention could be made and also still be within the scope of the invention. Consequently, it is not intended that the invention be limited to the specific embodiments and variants of the invention disclosed. It is to be further understood that changes and modifications to the descriptions given herein will occur to those skilled in the art. Therefore, the scope of the invention should be limited only by the scope of the claims.

What is claimed:

1. A lumen based pressure measuring guide wire system for measuring blood pressure within a body vessel, comprising:
a guide wire having a lumen with a distal opening and a proximal opening, the lumen being fillable with a fluid or other media, wherein the distal opening of the guide wire is adapted to be in fluid communication with the blood in the body vessel; and
a pressure measurement system in fluid communication with the proximal opening of the guide wire lumen which constantly fills the guide wire lumen with a fluid or media, the fluid or media exiting the guide wire lumen through the distal opening, wherein the pressure measurement system calculates the blood pressure being exerted at the distal opening of the guide wire lumen using the measured changing pressures in the measurement system as the fluid or media flows into the guide wire lumen.

2. The pressure measuring guide wire system of claim 1, wherein the measurement system includes:
a pressure transducer in fluid communication with the proximal opening of the guide wire lumen, the pressure transducer measuring pressure being exerted by the measurement system as the fluid or media is introduced into the guide wire lumen.

3. The pressure measuring guide wire system of claim 2, wherein the pressure measurement system further includes:
a processor in communication with the pressure transducer for calculating the blood pressure acting on the distal opening of the guide wire lumen using the measured changing pressures in the measurement system as the fluid or media flows into the guide wire lumen.

4. The pressure measuring guide wire system of claim 2, wherein the pressure measurement system further includes:
a compliant portion including compliant components in fluid communication with the pressure transducer and the guide wire lumen; and
a pump with a fluid reservoir in fluid communication with the pressure transducer and the compliant portion, wherein the compliant portion is injected with a volume of fluid or media by the pump that raises the pressure of the fluid or media in the pressure measuring system above the blood pressure in the body lumen causing a continuous flow of fluid or media into the guide wire lumen and out of the distal opening of the guide wire lumen.

5. The pressure measuring guide wire system of claim 4, wherein the changing pressures acting on the pressure transducer are communicated to the processor to calculate the blood pressure acting at the distal opening of the guide wire lumen.

6. The pressure measuring guide wire system of claim 5, wherein the processor is programmed to activate and deactivate the pump, the pump being configured to cause fluid or media to flow into the compliant portion and into the guide wire lumen.

7. The pressure measuring guide wire system of claim 4, wherein the processor is programmed with a lower limit pressure setting and a higher limit pressure setting, the processor being programmed to activate the pump to cause additional fluid or media to flow into the compliant portion when the pressure being measured by the processor at the pressure transducer falls below the lower limit pressure setting and to deactivate the pump when the upper limit pressure setting is reached.

8. The pressure measuring guide wire system of claim 1, wherein the internal lumen of the guide wire has a particular flow resistance (R) value and the measurement system has a particular compliance (C) value which is expressed as the property of the system to increase its volume in response to an increase in pressure exerted on the system, wherein the product of the flow resistance (R) value and the measurement system compliance (C) value defines a time constant (RC) value, the time constant (RC) value of the system being about one second or more.

9. The pressure measuring guide wire system of claim 3, wherein the internal lumen of the guide wire has a particular flow resistance (R) value and the measurement system has a particular compliance (C) value which is expressed as the property of the system to increase its volume in response to an increase in pressure exerted on the system, wherein the product of the flow resistance (R) value and the measurement system compliance (C) value defines a time constant (RC) value and the processor is programmable to calculate the time constant (RC) value.

10. A method for measuring blood pressure in a body vessel utilizing a guide wire having a lumen with a distal opening and a proximal opening, the guide wire lumen being in fluid communication with a measurement system that constantly fills the guide wire lumen with a fluid or other media, the measurement system being capable of introducing fluid into the proximal opening of the guide wire lumen which exits guide wire lumen through the distal opening, comprising:
placing the proximal opening of the guide wire lumen in fluid communication with the measurement system;
advancing the distal opening of the guide wire into the target location in the body vessel;
introducing fluid or other media via the measurement system into the proximal opening of the guide wire lumen while allowing the fluid or media to exit the guide wire lumen from the distal opening of the guide wire;
measuring the changing pressures in the measurement system as the fluid or media is being introduced into the proximal opening;
calculating the pressure at the distal opening of the guide wire lumen using the measured changing pressures of the measurement system.

11. The method of claim 10, wherein the pressure developed in the measurement system in introducing fluid or media into the guide wire lumen is greater than the blood pressure in the body vessel to cause the fluid or media to flow out of the distal opening of the guide wire lumen.

12. The method for measuring pressure in a body vessel of claim 11, further including:
introducing additional fluid or other media into the guide wire lumen when the measurement system detects that the measured pressure in the measurement system drops below a preset lower limit pressure.

13. The method for measuring pressure in a body vessel of claim 12, further including:
introducing fluid or other media into the guide wire lumen until the measured pressure in the measurement system reaches a preset upper limit pressure.

14. The method for measuring pressure in a body vessel of claim 13, further including:
stopping further introduction of fluid or other media into the internal lumen of the guide wire once the preset upper limit pressure has been reached.

15. The method of claim 10, wherein the internal lumen of the guide wire has a particular flow resistance (R) value and the measurement system has a particular compliance (C) value which is expressed as the property of the system to increase its volume in response to an increase in pressure exerted on the system, the product of the flow resistance (R) value and the measurement system compliance (C) value defining a time constant (RC) value, the method including:
calculating the value of the time constant (RC) of the system via the measurement system in a calibration step.

16. The method of claim 15, wherein the calculation of the value of the time constant (RC) of the system is performed before the distal opening of the guide wire lumen is inserted into the body vessel.

17. The method of claim 10, wherein the internal lumen of the guide wire has a particular flow resistance (R) value and the measurement system has a particular compliance (C) value which is expressed as the property of the system to increase its volume in response to an increase in pressure exerted on the system, the product of the flow resistance (R) value and the measurement system compliance (C) value defining a time constant (RC) value, the time constant (RC) value of the system being about one second or more.

18. The method of claim 15, wherein:
the measurement system includes a pressure transducer in fluid communication with the proximal opening of the guide wire lumen, the pressure transducer measuring pressure being exerted by the measurement system as the fluid or media is introduced into the guide wire lumen and a processor which receives signals from the pressure transducer that is in fluid communication with the guide wire lumen, the processor being in communication with the pump to control the pump.

19. The method of claim 18, wherein the pressure transducer and processor measures the changing pressures in the measurement system as the fluid or media is being introduced into the proximal opening;

20. The method of claim 19, wherein the measurement system further includes:
a compliant portion including compliant components in fluid communication with the pressure transducer and the guide wire lumen; and
a pump with a fluid reservoir in fluid communication with the pressure transducer and the compliant portion, wherein the compliant portion is injected with a volume of fluid or media by the pump that raises the pressure of the fluid or media in the pressure measuring system above the blood pressure in the body lumen causing a continuous flow of fluid or media into the guide wire lumen and out of the distal opening of the guide wire lumen.

21. The method of claim 20, wherein the internal lumen of the guide wire has a particular flow resistance (R) value and the measurement system has a particular compliance (C) value which is expressed as the property of the system to increase its volume in response to an increase in pressure exerted on the system, the product of the flow resistance (R) value and the measurement system compliance (C) value defining a time constant (RC) value, the method including:
calculating the value of the time constant (RC) of the system via the measurement system in a calibration step.

22. The method of claim 21, wherein the time constant (RC) of the system is about one second or more.

23. A lumen based pressure measuring guide wire system for measuring pressure within a body vessel, comprising:
a guide wire having a lumen with a distal opening and a proximal opening, the lumen being fillable with a fluid or other media, wherein the distal opening of the guide wire is adapted to be in fluid communication with the blood in the body vessel; and
a pressure measurement system in fluid communication with the proximal opening of the guide wire lumen which constantly fills the guide wire lumen with a fluid or media, the fluid or media exiting the guide wire lumen through the distal opening, wherein the pressure measurement system calculates the blood pressure being exerted at the distal opening of the guide wire lumen using the measured changing pressures in the measurement system as the fluid or media flows into the guide wire lumen, the pressure measurement system including:
a pressure transducer in fluid communication with the proximal opening of the guide wire lumen, the pressure transducer measuring pressure being exerted by the measurement system as the fluid or media is introduced into the guide wire lumen;
a processor in communication with the pressure transducer for calculating the blood pressure acting on the distal opening of the guide wire lumen using the measured changing pressures in the measurement system as the fluid or media flows into the guide wire lumen;
a compliant portion including compliant components in fluid communication with the pressure transducer and the guide wire lumen; and
a pump with a fluid reservoir in fluid communication with the pressure transducer and the compliant portion, wherein the compliant portion is injected with a volume of fluid or media by the pump that raises the pressure of the fluid or media in the pressure measuring system above the blood pressure in the body lumen causing a continuous flow of fluid or media into the guide wire lumen and out of the distal opening of the guide wire lumen.

24. The pressure measuring guide wire system of claim 23, wherein the changing pressures acting on the pressure transducer are communicated to the processor to calculate the blood pressure acting at the distal opening of the guide wire lumen.

25. The pressure measuring guide wire system of claim 24, wherein the processor is programmed to activate and deactivate the pump, the pump being configured to cause fluid or media to flow into the compliant portion and into the guide wire lumen.

26. The pressure measuring guide wire system of claim 23, wherein the internal lumen of the guide wire has a particular flow resistance (R) value and the measurement system has a particular compliance (C) value which is expressed as the property of the system to increase its volume in response to an increase in pressure exerted on the system, wherein the product of the flow resistance (R) value and the measurement system compliance (C) value defines a time constant (RC) value, the time constant (RC) value of the system being about one second or more.

27. The pressure measuring guide wire system of claim 23, wherein the internal lumen of the guide wire has a particular flow resistance (R) value and the measurement system has a particular compliance (C) value which is expressed as the property of the system to increase its volume in response to an increase in pressure exerted on the system, wherein the product of the flow resistance (R) value and the measurement system compliance (C) value defines a time constant (RC) value and the processor is programmable to calculate the time constant (RC) value.

28. A method for calculating blood pressure in a body vessel utilizing a guide wire having a lumen with a distal opening and a proximal opening, the proximal opening of the guide wire lumen being in fluid communication with a measurement system that constantly fills the guide wire lumen with a fluid or other media, the measurement system being capable of introducing fluid into the proximal opening of the guide wire lumen which exits the guide wire lumen through the distal opening, a pressure transducer in fluid communication with the proximal opening of the guide wire lumen, the pressure transducer measuring pressure being exerted by the measurement system as the fluid or media is introduced into the guide wire lumen, a processor in communication with the pressure transducer for calculating the blood pressure acting on the distal opening of the guide wire lumen using the measured changing pressures in the measurement system as the fluid or media flows into the guide wire lumen, a compliant portion including compliant components in fluid communication with the pressure transducer and the guide wire lumen, and a pump with a fluid reservoir in fluid communication with the pressure transducer and the compliant portion, the method comprising:
placing the proximal opening of the guide wire lumen in fluid communication with the measurement system;
advancing the distal opening of the guide wire into the target location in the body vessel;
introducing fluid or other media into the compliant portion that raises the pressure of the fluid or media in the pressure measuring system above the highest blood pressure in the body lumen causing a continuous flow of fluid or media into the guide wire lumen and out of the distal opening of the guide wire lumen;
measuring the changing pressures at the pressure transducer as the fluid or media is being introduced into the proximal opening; and
calculating the pressure at the distal opening of the guide wire lumen via the processor using the obtained measured changing pressures at the pressure transducer as the fluid or media is being introduced into the proximal opening of the guide wire lumen.

29. The method of claim 28, wherein the guide wire lumen has a particular flow resistance (R) value and the measurement system has a particular compliance (Cₓ) value which is expressed as the property of the system to increase its volume in response to an increase in pressure exerted on the system, wherein the product of the flow resistance (R) value and the measurement system compliance (Cₓ) values define time constant (RCₓ) values, the method further including:
calculating the time constant (RCₓ) values of the measurement system.

30. The method of claim 29, further including:
storing the calculated time constant (RCₓ) values of the measurement system in the processor.

31. The method of claim 29, wherein the time constant (RCₓ) values are calculated using the following equation:
$${RC}_{x} = \frac{{t}_{x + 1} - {t}_{x}}{ln ( \frac{{P}_{x}}{{P}_{x + 1}} )}$$
where:
R=Flow resistance of the guide wire lumen to the fluid flow
Cₓ=Compliance of the (compliant) system (at the proximal end of the guide wire lumen) during sample time interval (tₓ₊₁−tₓ)
Pₓ=The measured pressure in the (compliant) system at time tₓ
tₓ=Sequential sample time measurement number x after the upper pressure limit is attained/fluid injection into the compliant system is stopped.

32. The method of claim 31, wherein prior to the placement of the distal opening of the guide wire lumen into the body vessel, changing pressures are measured by the pressure transducer while the distal opening of the guide wire lumen is exposed to atmospheric pressure and the time constant (RCₓ) values are calculated using the measured pressure data and the pressure measurement data sampling time interval.

33. The method of claim 31, wherein prior to the placement of the distal opening of the guide wire lumen into the body vessel, changing pressures are measured by the pressure transducer while the distal opening of the guide wire lumen is submerged a short distance in a wetting bath and the time constant (RCₓ) values are calculated using the measured pressure data and the pressure measurement data sampling time interval.

34. The method of claim 32, wherein the calculation of the blood pressure in the body vessel is performed using the detected pressure data, the recorded (RCₓ) time constant data and their data sampling time intervals.

35. The method of claim 30, wherein prior to the placement of the distal opening of the guide wire lumen into the body vessel, the measurement system is activated for several cycles to stabilize the (RCₓ) values

36. The method of claim 28, wherein the measuring of pressure at the pressure transducer as the fluid or media is being introduced into the proximal opening of the guide wire lumen is terminated after a preset time period.

37. The method of claim 28, wherein the measuring of pressure at the pressure transducer as the fluid or media is being introduced into the proximal opening of the guide wire lumen is terminated after the pressure transducer measures a particular pressure.
